(19) United States
(12) Patent Application Publication
(10) Pub. No.: US 2024/0352061 A1
(43) Pub. Date: Oct. 24, 2024

(54) PROCESS FOR SEPARATION AND QUANTIFICATION OF NON-IONIC SURFACTANT

(71) Applicant: Kashiv BioSciences, LLC, Piscataway, NJ (US)

(72) Inventors: Shalini Sharma, Piscataway, NJ (US); Hetal Katrodiya, Ahmedabad (IN); Vivek Dave, Ahmedabad (IN)

(21) Appl. No.: 18/758,622

(22) Filed: Jun. 28, 2024

Related U.S. Application Data
(63) Continuation of application No. PCT/IB2022/062943, filed on Dec. 31, 2022.

(30) Foreign Application Priority Data
Dec. 31, 2021 (IN) 202121062108

(51) Int. Cl. C07K 1/20; G01N 30/72; G01N 30/74; G01N 30/88

(52) U.S. Cl. CPC C07K 1/20; G01N 30/72; G01N 30/74; G01N 30/88; G01N 2030/884

(57) ABSTRACT

The present invention relates to a separation and quantification method of a non-ionic surfactant in a composition comprising polypeptide and non-ionic surfactant by using a zwitterionic hydrophilic interaction liquid chromatography (ZIC-HILIC).

### FIELD OF INVENTION

The present invention relates to a separation and quantification method of a non-ionic surfactant in a composition comprising polypeptide and non-ionic surfactant by using a zwitterionic hydrophilic interaction liquid chromatography (ZIC-HILIC).

### BACKGROUND OF THE INVENTION

Biopharmaceutical formulation contains surfactant that owes excellent protein-stabilizing properties by limiting the air liquid interaction with protein and resembles good safety profiles.

Surfactants commonly used in biotherapeutic formulation prevents physical damage to polypeptide during operational steps. Polysorbates and Poloxamers are surfactants appears to be a promising to eliminate or minimize the adverse effects associated with the molecule. The high amount of polysorbate in formulation is linked to developmental and reproductive toxicity, immunotoxicity and allergies and another side the low amount of polysorbate in formulation minimizes the risk of damage to a protein product and being safe for human use. The optimal quantity during the stability and release testing is often required by regulatory bodies such as FDA.

The HPLC-based methods are used for separation and relative quantitation of polysorbate or other excipients with an appropriately established reference material to prevent unreasonable and significant risk of illness or injury to human subjects and to provide sufficient information to assess risk to human subjects.

The conventional quantification and separation process of non-ionic surfactant in biopharmaceutical formulation is susceptible to have interference of protein specifically hydrophobic proteins. Some technique utilizes certain steps of pretreatment like protein precipitation etc. where accuracy of the quantification is the question. Therefore, there is a need of a process to provide high resolution separation, identification, and quantification of the various components such as non-ionic surfactant from the sample mixture. Further, the process provides an advantageous selectivity towards non-ionic surfactant using normal phase chromatography column addressed in present invention.

The current invention discusses separation and quantification of non-ionic surfactant in biotherapeutic drug product by using a zwitterionic hydrophilic interaction liquid chromatography (ZIC-HILIC) column. The method is effective to separate non-ionic surfactant and protein which is useful to assure product stability, and final release.

### SUMMARY OF THE INVENTION

The present invention pertains a separation and quantification method for a non-ionic surfactant in a composition comprising polypeptide and non-ionic surfactant by using a zwitterionic hydrophilic interaction liquid column chromatography.

In an embodiment, a method for separation of non-ionic surfactant from a protein mixture comprising protein of interest and non-ionic surfactant comprising;
a) loading the protein mixture on zwitterionic hydrophilic interaction liquid chromatography (ZIC-HILIC) column;
b) eluting the non-ionic surfactant from ZIC-HILIC column by using mobile phase A and B in a suitable ratio;
c) eluting the protein of interest from ZIC-HILIC column by using mobile phase A and B in a suitable ratio;
wherein the protein mixture is a biopharmaceutical formulation or composition, and the non-ionic surfactant elutes before the elution of protein of interest.

In an embodiment, a method for separation and quantification of non-ionic surfactant from a protein mixture comprising protein of interest and non-ionic surfactant comprising;
a) loading the protein mixture on zwitterionic hydrophilic interaction liquid chromatography (ZIC-HILIC) column;
b) eluting the non-ionic surfactant from ZIC-HILIC column by using mobile phase A and B in a suitable ratio;
c) eluting the protein of interest from ZIC-HILIC column by using mobile phase A and B in a suitable ratio;
d) quantifying the non-ionic surfactant using a detection technique;
wherein the protein mixture is a biopharmaceutical formulation or composition, and the non-ionic surfactant elutes before the elution of protein of interest.

In an embodiment, the eluted non-ionic surfactant in step (b) quantified by suitable technique selected from ultraviolet light absorbance (UV), fluorescence (FL), refractive index (RI), evaporative light scattering (ELSD), charged aerosol (CAD), and mass spectrometry (MS).

In an embodiment, a method for improving the resolution of non-ionic surfactant in protein mixture comprising;
a) loading the protein mixture comprising protein of interest and non-ionic surfactant on zwitterionic hydrophilic interaction liquid chromatography (ZIC-HILIC) column;
b) eluting the non-ionic surfactant from the zwitterionic hydrophilic interaction liquid chromatography (ZIC-HILIC) column;
wherein the elution of non-ionic surfactant is performed at temperature more than about 50° C. to about 65° C.

In an embodiment, a zwitterionic hydrophilic interaction liquid chromatography (ZIC-HILIC) column for the separation and quantification of a non-ionic surfactant in biopharmaceutical formulation comprises:
a) loading the biopharmaceutical composition onto the ZIC-HILIC column;
b) eluting the non-ionic surfactant by passing the mobile phase A and mobile phase B through the column;
c) measuring the first void peak of eluted sample coupled to a detection technique.
wherein the eluted sample is a non-ionic surfactant.

In an embodiment, the non-ionic surfactant selected from polyglycerol alkyl ethers, glucosyl dialkyl ethers, crownethers, ester-linked surfactants, polyoxyethylene alkyl ethers, BYK-110, Tween-20 (Polysorbate 20 or PS20), Tween-80 (Polysorbate 80), Poloxamer 188 (P188) and Nonidet-P40 (nonylphenoxypolyethoxyethanol).

In an embodiment, the mobile phase A or mobile phase B is an organic solvent, or water and/or combination thereof to improve the binding affinity of protein of interest with column resin.

In an embodiment, the mobile phase A or mobile phase B is an organic solvent, or water and/or combination thereof to improve the elution of protein of interest.

### BRIEF DESCRIPTION OF FIGURES

FIG. 1 depicts the void peak of non-ionic surfactant PS20 from ZIC-HILIC column.

FIG. 2 depicts the efficient separation of non-ionic surfactant PS20 and polypeptide. The quantification profile as void peak of eluted non-ionic surfactant PS20 before the polypeptide peak using a ZIC-HILIC column.

FIG. 3 depicts the standard peak of non-ionic surfactant PS20 and protein sample containing antibody without non-ionic surfactant PS20.

FIG. 4 depicts the overlay of standard peak of non-ionic surfactant PS20 with non-ionic surfactant PS20 in a protein sample containing antibody with non-ionic surfactant PS20.

FIG. 5 depicts the overlay of standard peak of non-ionic surfactant P188 and protein sample containing antibody without non-ionic surfactant P188.

FIG. 6 depicts the separation and quantification profile of non-ionic surfactant P188 and protein sample containing antibody. The overlay of standard peak of non-ionic surfactant P188 and P188 peak prior protein sample containing antibody peak with non-ionic surfactant P188.

FIG. 7 depicts the separation and quantification profile of non-ionic surfactant P188 and protein sample containing fusion protein. The overlay of standard peak of non-ionic surfactant P188 with peak of non-ionic surfactant P188 in a protein sample containing fusion protein with non-ionic surfactant P188.

FIG. 8 depicts the effect of column temperature 40° C. and 55° C. the overlay of Poloxamer 188 peak the peak width increased when temperatures decreased.

FIG. 9 depicts the effect of column temperature 55° C. and 65° C. for working operation.

FIG. 10 depicts the Linearity curve (Quadratic calibration curve) between average peak area (pA*min) and Poloxamer 188 concentration (mg/mL) FIG. 11 depicts the Linearity curve (Quadratic calibration curve) between average peak area (pA*min) and Polysorbate 20 concentration (mg/mL)

### DETAILED DESCRIPTION OF INVENTION

The present invention pertains to a separation and/or quantification method for a non-ionic surfactant in a composition comprising polypeptide and non-ionic surfactant by employing a zwitterionic hydrophilic interaction liquid column chromatography.

### Definitions

The term “comprises” or “comprising” is used in the present description, it does not exclude other elements or steps. For purpose of the present invention, the term “consisting of’ is considered to be an optional embodiment, of the term “comprising of’. If hereinafter a group is defined to comprise at least a certain number of embodiments, this is also to be understood to disclose a group which optionally consists only of these embodiments. As used throughout the specification and in the appended claims, the singular forms “a,” “an,” and “the” include the plural reference unless the context clearly dictates otherwise.

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

The term “polypeptide” or “protein” or “antibody or monoclonal antibody” or “fusion protein” are interchangeable refers to polymers of amino acids of any length. The separation of protein from a biopharmaceutical formulation elutes after the elution of non-ionic surfactant in chromatography column.

In an embodiment, antibodies are selected from IgG1, IgG2, IgG3, IgG4 and fusion proteins. In certain embodiments the antibodies and fusion proteins are selected from Rituximab, Palivizumab, Etanercept, Abatacept, Aflibercept, Belatacept, Rilonacept, Romiplostim, Alefacept, Conbercept, Infliximab, Trastuzumab, Alemtuzumab, Adalimumab, Ibritumomab tiuxetan, Omalizumab, Cetuximab, Bevacizumab, Natalizumab, Eculizumab, Certolizumab pegol, Ustekinumab, Canakinumab, Golimumab, Ofatumumab, Tocilizumab, Denosumab, Belimumab, Ipilimumab, Brentuximab vedotin, Pertuzumab, Trastuzumab emtansine, Raxibacumab, Obinutuzumab, Siltuximab, Ramucimmab, Vedolizumab, Blinatumomab, Nivolumab, Pembrolizumab, Darucizumab, Necitumumab, Dinutuximab, Secukinumab, Mepolizumab, Alirocumab, Evolocumab, Daratumumab, Elotuzumab, Ixekizumab, Reslizumab, Olaratumab, Bezlotoxumab, Atezolizumab, Obiltoxaximab, Sarilumab, Ocrelizumab, Tildrakizumab, Romosozumab, Brolucizumab, and Crizanlizumab.

Omalizumab (anti-IgE antibody) is a recombinant DNA-derived humanized IgGlK monoclonal antibody that selectively binds to human immunoglobulin (IgE). The antibody has a molecular weight of approximately 149 kD.

Abatacept is a soluble fusion protein that consists of the extracellular domain of human cytotoxic T-lymphocyte-associated antigen 4 (CTLA-4) linked to the modified Fc (hinge, CH2, and CH3 domains) portion of human immunoglobulin G1 (IgG1). Abatacept is produced by recombinant DNA technology in a mammalian cell expression system. The molecular weight of abatacept is 92 kilodaltons.

The term “Hydrophilic interaction liquid chromatography” or “HILIC” refers to a process used for effective separation of polar compounds on polar stationary phases shows the specific and non-specific binding interaction. HILIC is a variant of normal phase liquid chromatography for the separation of complex peptide mixtures, and it can be combined with several detection techniques, such as ultraviolet light absorbance (UV), fluorescence (FL), refractive index (RI), evaporative light scattering (ELSD), charged aerosol (CAD), and mass spectrometry (MS) further HILIC described in art (Anal Bioanal Chem. 2012; 402(1): 231-247).

A zwitterion compounds holds both positive and negative charges, on its surface the distribution of positive and negative charges is paralleled to the surface of the solid matrix on zwitterionic HILIC stationary phase prepared from compound with zwitterionic functional group and can be optimized and adjusted by changing the pH values. The “ZIC-HILIC” chromatography column have high polarity and good hydrophilicity for hydrophilic interaction chromatographic stationary phase and fulfills the requirement of unique selectivity mediated separation of molecules.

As used herein the term “column” or “resin” or “chromatographic resin or chromatographic column” are interchangeable.

The term “Quantification” or “Estimation” or “Quantitation” are interchangeable refers to the measurement of peak value by detector coupled to chromatography column demonstrates the quantification of surfactant in biopharmaceutical formulation or composition.

The term “Specificity” used herein refers to the ability of the method to assess the analyte unequivocally in the presence of components which may be expected to be present in a test sample.

The term “Linearity” used herein refers to any analytical procedure is its ability (within a given range) to obtain test results which are directly proportional to the concentration (amount) of analyte in the sample.

The term “Repeatability” or “intra-assay precision” used herein refers to the ability of the method to generate the same results over a short period of time under identical conditions.

The term “Accuracy” used herein refers to analytical procedure expresses the closeness of agreement between the value, which is accepted either as a conventional true value or an accepted reference value and the value found.

The term “Biopharmaceutical formulation” or “Liquid formulation” refers to formulations comprising proteins or monoclonal antibodies or fusion proteins and a variety of excipients to maintain pH and tonicity and to increase protein stability and preservation. As used herein “protein mixture” or “protein sample” or “Biopharmaceutical formulation” or “Liquid formulation” used herein are interchangeable and refers to the solutions containing protein of interest along with non-ionic surfactant. As used herein “diluent” refers to protein sample that is prepared in the solution selected from formic acid (FA), methanol, acetonitrile (ACN), ethanol, acetic acid, trifluoroacetic acid (TFA) isopropanol (IPA) and water.

The term “non-ionic surfactant” includes the separation and quantification of surfactants selected from golyglycerol alkyl ethers, glucosyl dialkyl ethers, crownethers, ester-linked surfactants, polyoxyethylene alkyl ethers, BYK-110, Polysorbate 20 (PS 20), Polysorbate 40 (PS 40), Polysorbate (PS 60), Polysorbate 80 (PS 80), Poloxamer 188 (P188), Poloxamer 237 (P237), Poloxamer (P338), Poloxamer (P407) and Nonidet-P40 (nonylphenoxypolyethoxyethanol).

The term “mobile phase” or “eluent” refers to solution that run throughout the column into which stationary phase is fixed and the separation of mixture or composition or sample occurs. In an embodiment, the mobile phase used herein capable to separate dissolved components (e.g., surfactant and/or polypeptide) present in the protein mixture. The term “mobile phase” used herein essentially consisting of mobile phase A and mobile phase B. The mobile phase A and mobile phase B is interchangeable with “first buffer” or “second buffer” respectively in ZIC-HILIC. The mobile phase A and mobile phase B is used in suitable ratio to form a gradient in order to perform separation of non-ionic surfactant and protein of interest.

The mobile phase is selected from organic solvent or water and/or combinations thereof. The organic solvent is selected from formic acid, methanol, acetonitrile, ethanol, acetic acid, trifluoroacetic acid (TFA), and isopropanol. The mobile phase A or mobile phase B is an organic solvent, or water and/or combination thereof to improve the binding affinity of protein of interest with column resin. The mobile phase selected for improving the binding affinity of protein of interest with column resin and/or eluting the non-ionic surfactant comprises substantial amount of organic solvent in comparison to aqueous solution such as water. The aqueous solution or water is present less than 10%, preferably about 5%. The amount of organic solvent is about 90%, preferably 95%.

The mobile phase selected for reducing the binding affinity of protein of interest with column resin comprises substantial amount of aqueous solution such as water and in comparison, to organic solvent. The aqueous solution or water is present more than about 95%, preferably more than 99%. The amount of organic solvent is about 5%, preferably about 0.1%.

Any skilled person can select mobile phase A and mobile phase B in view of the teaching of the present disclosure to select either mobile phase A or mobile phase B for eluting non-ionic surfactant or for the binding of protein of interest to the column.

In an embodiment, the mobile phase A or mobile phase B is an organic solvent, or water and/or combination thereof to improve the elution of protein of interest.

In an embodiment, the mobile phase A is an organic solvent selected from formic acid, methanol, acetonitrile, ethanol, acetic acid, trifluoroacetic acid (TFA), isopropanol, and water and/or combination thereof.

In an embodiment, the concentration of formic acid is selected from about 0.1%, about 0.2%, about 0.3%, about 0.4%, about 0.5%, about 0.6%, about 0.7%, about 0.8%, about 0.9% and about 1%.

In another embodiment, the concentration of formic acid is about 0.1%.

In an embodiment, the concentration of methanol is selected from about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%.

In another embodiment, the concentration of methanol is about 35%.

In an embodiment, the concentration of acetonitrile is selected from about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%.

In another embodiment, the concentration of acetonitrile is about 60%.

In an embodiment, the concentration of water is selected from about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, and 99.9%.

In an embodiment, the concentration of water is about 5%.

In an embodiment, the mobile phase B is selected from methanol, acetonitrile, ethanol, isopropanol. formic acid and water and/or combination thereof.

In an embodiment, the concentration of formic acid is selected from about 0.1%, about 0.2%, about 0.3%, about 0.4%, about 0.5%, about 0.6%, about 0.7%, about 0.8%, about 0.9% and about 1%.

In another embodiment, the concentration of formic acid is about 0.1%.

In an embodiment, the concentration of methanol is selected from about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%.

In an embodiment, the concentration of isopropanol is selected from about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%.

In an embodiment, the concentration of acetonitrile is selected from about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%.

In an embodiment, the concentration of water is selected from about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, and 99.9%.

In an embodiment, the concentration of water is 99.9%.

In an embodiment the mobile phase B comprises formic acid in concentration about 0.1% and water in concentration more than 99% preferably 99.9%.

In an embodiment, the mobile phase A is an organic solvent, or water and/or combination thereof and mobile phase B is formic acid and water and/or combination thereof.

In an embodiment, the elution of non-ionic surfactant is performed by using mobile phase A in substantially higher amount than mobile phase B, wherein the ratio of mobile phase A to mobile phase B maintained about 100:0, about 90:10, about 80:20, and about 70:30. Skilled person can optimize the ratio to avoid the elution of protein of interest with non-ionic surfactant.

In an embodiment, the elution of protein of interest is performed by using mobile phase B in substantially higher amount than mobile phase A, wherein the ratio of mobile phase B to mobile phase A maintained about 30:70, about 20:80, about 10:90, and about 0:100.

The term “flow rate” refers to amount of mobile phase passing through the column in unit time.

The term “void volume” or “dead volume” refers to the volume of mobile phase required to elute a molecule that has zero retention in the stationary phase. The volume of the liquid phase contained inside a column from time of injection to its detection via detector in a chromatography column as it is unretained in the stationary phase. In an embodiment, “early peak” or “non-ionic surfactant peak” used herein as elution of non-ionic surfactant prior to the protein of interest from the chromatography column detected through a detection technique selected from ultraviolet light absorbance (UV), fluorescence (FL), refractive index (RI), evaporative light scattering (ELSD), charged aerosol (CAD), and mass spectrometry (MS).

In an embodiment, a method for separation of non-ionic surfactant from a protein mixture comprising protein of interest and non-ionic surfactant comprising;
a) loading the protein mixture on zwitterionic hydrophilic interaction liquid chromatography (ZIC-HILIC) column;
b) eluting the non-ionic surfactant from ZIC-HILIC column by using mobile phase A and B in a suitable ratio;
c) eluting the protein of interest from ZIC-HILIC column by using mobile phase A and B in a suitable ratio;
wherein the protein mixture is a biopharmaceutical formulation or composition, and the non-ionic surfactant elutes before the elution of protein of interest.

In an embodiment, a method for separation and quantification of non-ionic surfactant from a protein mixture comprising protein of interest and non-ionic surfactant comprising; a) loading the protein mixture on zwitterionic hydrophilic interaction liquid chromatography (ZIC-HILIC) column;
b) eluting the non-ionic surfactant from ZIC-HILIC column by using mobile phase A and B in a suitable ratio;
c) eluting the protein of interest from ZIC-HILIC column by using mobile phase A and B in a suitable ratio;
d) quantifying the non-ionic surfactant using a detection technique;
wherein the protein mixture is a biopharmaceutical formulation or composition, and the non-ionic surfactant elutes before the elution of protein of interest.

In an embodiment, the eluted non-ionic surfactant in step (b) quantified by suitable technique selected from ultraviolet light absorbance (UV), fluorescence (FL), refractive index (RI), evaporative light scattering (ELSD), charged aerosol (CAD), and mass spectrometry (MS).

In an embodiment, the elution of non-ionic surfactant in step (b) is performed by using mobile phase A in substantially higher amount than mobile phase B, wherein the ratio of mobile phase A to mobile phase B is selected from about 100:0, about 90:10, about 80:20, and about 70:30.

In an embodiment, the elution of protein of interest in step (c) by using mobile phase B in substantially higher amount than mobile phase A, wherein the ratio of mobile phase B to mobile phase A is selected from about 30:70, about 20:80, about 10:90, and about 0:100.

In an embodiment, a method for improving the resolution of non-ionic surfactant in protein mixture comprising;
c) loading the protein mixture comprising protein of interest and non-ionic surfactant on zwitterionic hydrophilic interaction liquid chromatography (ZIC-HILIC) column;
d) eluting the non-ionic surfactant from the zwitterionic hydrophilic interaction liquid chromatography (ZIC-HILIC) column;
wherein the elution of non-ionic surfactant is performed at temperature more than about 50° C. to about 65° C.

In an embodiment, a method for separation and quantification of a non-ionic surfactant in a composition comprising;
a) loading the non-ionic surfactant and polypeptide composition on zwitterionic hydrophilic interaction liquid chromatography (ZIC-HILIC) column;
b) eluting the non-ionic surfactant from ZIC-HILIC column by passing mobile phase A and B;
c) eluting the polypeptide from ZIC-HILIC column by passing mobile phase A and B; d) quantifying the non-ionic surfactant using a detection technique;
wherein non-ionic surfactant elutes before the elution of said polypeptide and shows an early peak in chromatogram.

In an embodiment, a method for separation and quantification of Polysorbate 20 or PS20 in a composition comprising;
a) loading the PS20 and anti-IgE antibody in a composition on zwitterionic hydrophilic interaction liquid chromatography (ZIC-HILIC) column;
b) eluting the non-ionic PS20 from ZIC-HILIC column with mobile phase A and B;
c) eluting anti-IgE antibody from ZIC-HILIC column with mobile phase A and B;
d) quantifying the PS20 using a detection technique;
wherein non-ionic surfactant elutes before the elution of said polypeptide and shows an early peak in chromatogram.

In an embodiment, a method for separation and quantification of Poloxamer 188 or P188 in a composition comprising;
a) loading the PS20 and anti-IgE antibody in a composition on zwitterionic hydrophilic interaction liquid chromatography (ZIC-HILIC) column;
b) eluting the non-ionic PS20 from ZIC-HILIC column with mobile phase A and B;
c) eluting anti-IgE antibody from ZIC-HILIC column with mobile phase A and B;
d) quantifying the PS20 using a detection technique;
wherein non-ionic surfactant elutes before the elution of said polypeptide and shows an early peak in chromatogram.

In an embodiment, a method for separation and quantification of Polysorbate 20 or PS20 in a composition comprising;
a) loading the PS20 and monoclonal antibody in a composition on zwitterionic hydrophilic interaction liquid chromatography (ZIC-HILIC) column;
b) eluting the non-ionic PS20 from ZIC-HILIC column with mobile phase A and B;
c) eluting monoclonal antibody from ZIC-HILIC column with mobile phase A and B;
d) quantifying the PS20 using a Charged Aerosol Detector (CAD);
wherein non-ionic surfactant elutes before the elution of said polypeptide and shows an early

In an embodiment, a method for separation and quantification of non-ionic surfactant Poloxamer 188 (P188) in a composition comprising;
a) loading the non-ionic surfactant P188 and fusion protein in a composition on zwitterionic hydrophilic interaction liquid chromatography (ZIC-HILIC) column;
b) eluting the non-ionic surfactant P188 from ZIC-HILIC column with mobile phase A and B in suitable ratio;
c) eluting fusion protein from ZIC-HILIC column with mobile phase A and B in suitable ratio;
d) quantifying the non-ionic surfactant P188 using a Charged Aerosol Detector (CAD);
wherein non-ionic surfactant P188 elutes before the elution of fusion protein and shows an early peak in chromatogram.

In an embodiment, a method for separation and quantification of non-ionic surfactant Polysorbate 20 (PS20) in a composition comprising;
a) loading the non-ionic surfactant PS20 and fusion protein in a composition on zwitterionic hydrophilic interaction liquid chromatography (ZIC-HILIC) column;
b) eluting the non-ionic surfactant PS20 from ZIC-HILIC column with mobile phase A and B in suitable ratio;
c) eluting fusion protein from ZIC-HILIC column with mobile phase A and B in suitable ratio;
d) quantifying the non-ionic surfactant PS20 using a Charged Aerosol Detector (CAD);
wherein non-ionic surfactant PS20 elutes before the elution of fusion protein and shows an early peak in chromatogram.

In an embodiment, a method for separation and quantification of non-ionic surfactant from a protein mixture comprising monoclonal antibody and non-ionic surfactant comprising; a) loading the protein mixture on zwitterionic hydrophilic interaction liquid chromatography (ZIC-HILIC) column;
b) eluting the non-ionic surfactant from ZIC-HILIC column by using mobile phase A and B in a suitable ratio;
c) eluting the monoclonal antibody from ZIC-HILIC column by using mobile phase A and B in a suitable ratio;
d) quantifying the non-ionic surfactant using a detection technique;
wherein the protein mixture is a biopharmaceutical formulation or composition, and the non-ionic surfactant elutes before the elution of monoclonal antibody.

In an embodiment, a method for separation and quantification of non-ionic surfactant from a protein mixture comprising fusion protein and non-ionic surfactant comprising;
a) loading the protein mixture on zwitterionic hydrophilic interaction liquid chromatography (ZIC-HILIC) column;
b) eluting the non-ionic surfactant from ZIC-HILIC column by using mobile phase A and B in a suitable ratio;
c) eluting the fusion protein from ZIC-HILIC column by using mobile phase A and B in a suitable ratio;
d) quantifying the non-ionic surfactant using a detection technique;
wherein the protein mixture is a biopharmaceutical formulation or composition, and the non-ionic surfactant elutes before the elution of fusion protein.

In an embodiment, a method for separation and quantification of Polysorbate 20 (PS20) from a protein mixture comprising anti-IgE antibody and Polysorbate 20 (PS20) comprising;
a) loading the protein mixture on zwitterionic hydrophilic interaction liquid chromatography (ZIC-HILIC) column;
b) eluting the Polysorbate 20 (PS20) from ZIC-HILIC column by using mobile phase A and B in a suitable ratio;
c) eluting anti-IgE antibody from ZIC-HILIC column by using mobile phase A and B in a suitable ratio;
d) quantifying the Polysorbate 20 (20) using a detection technique;
wherein the protein mixture is a biopharmaceutical formulation or composition, and the Polysorbate 20 (PS20) elutes before the elution of anti-IgE antibody.

In an embodiment, a method for separation and quantification of Poloxamer 188 (P188) from a protein mixture comprising anti-IgE antibody and Poloxamer 188 (P188) O comprising;
a) loading the protein mixture on zwitterionic hydrophilic interaction liquid chromatography (ZIC-HILIC) column;
b) eluting the Poloxamer 188 (P188) from ZIC-HILIC column by using mobile phase A and B in a suitable ratio;
c) eluting anti-IgE antibody from ZIC-HILIC column by using mobile phase A and B in a suitable ratio;
d) quantifying the Poloxamer 188 (P188) using a detection technique;
wherein the protein mixture is a biopharmaceutical formulation or composition, and the Poloxamer 188 (P188) elutes before the elution of anti-IgE antibody.

In an embodiment, a method for separation and quantification of Poloxamer 188 (P188) from a protein mixture comprising CTLA4-Ig fusion protein and Poloxamer 188 (P188) comprising;
a) loading the protein mixture on zwitterionic hydrophilic interaction liquid chromatography (ZIC-HILIC) column;
b) eluting the Poloxamer 188 (P188) from ZIC-HILIC column by using mobile phase A and B in a suitable ratio;
c) eluting the CTLA4-Ig fusion protein from ZIC-HILIC column by using mobile phase A and B in a suitable ratio;
d) quantifying the Poloxamer 188 (P188) using a detection technique;
wherein the protein mixture is a biopharmaceutical formulation or composition, and the P188 elutes before the elution of CTLA4-Ig fusion protein.

In an embodiment, a method for separation and quantification of Polysorbate 20 (PS20) from a protein mixture comprising CTLA4-Ig fusion protein and Polysorbate 20 (PS20) comprising;
a) loading the protein mixture on zwitterionic hydrophilic interaction liquid chromatography (ZIC-HILIC) column;
b) eluting the Polysorbate 20 (PS20) from ZIC-HILIC column by using mobile phase A and B in a suitable ratio;
c) eluting the CTLA4-Ig fusion protein from ZIC-HILIC column by using mobile phase A and B in a suitable ratio;
d) quantifying the Polysorbate 20 (PS20) using a detection technique;
wherein the protein mixture is a biopharmaceutical formulation or composition, and the PS20 elutes before the elution of CTLA4-Ig fusion protein.

In an embodiment, the biopharmaceutical formulation comprises about 150 mg/ml anti-IgE antibody, and 0.4 mg/ml Poloxamer 188 with other excipients such as buffer, amino acid, sugars, polyols, tonicity agents etc.

In an embodiment, the biopharmaceutical formulation comprises about 150 mg/ml anti-IgE antibody, and 0.4 mg/ml Polysorbate 20 with other excipients such as buffer, amino acid, sugars, polyols, tonicity agents etc.

In an embodiment, the biopharmaceutical formulation comprises about 125 mg/ml CTLA4-Ig fusion protein, and 8 mg/ml Poloxamer 188 with other excipients such as buffer, amino acid, sugars, polyols, tonicity agents etc.

In certain embodiment, the protein concentration containing more than 150 mg/ml of protein and low concentration of non-ionic surfactant in biopharmaceutical formulation the pretreatment is required.

In another embodiment, the non-ionic surfactant selected from golyglycerol alkyl ethers, glucosyl dialkyl ethers, crownethers, ester-linked surfactants, polyoxyethylene alkyl ethers, BYK-110, Tween-20 (Polysorbate 20), Tween-80 (Polysorbate 80), Poloxamer 188 and Nonidet-P40 (nonylphenoxypolyethoxyethanol).

In an embodiment, the non-ionic surfactant is Polysorbate 20 or PS20.

In an embodiment, the non-ionic surfactant is Poloxamer 188 or P188.

In an embodiment, the protein of interest is selected from peptide, antibody, antibody fragment, pegylated protein and fusion protein.

In an embodiment, detection of non-ionic surfactant using the detection techniques selected from ultraviolet light absorbance (UV), fluorescence (FL), refractive index (RI), evaporative light scattering (ELSD), charged aerosol (CAD), and mass spectrometry (MS).

In an embodiment, the detection technique is charged aerosol detector (CAD).

In certain embodiment, the mobile phase A comprises about 0.1% formic acid, about 35% methanol, about 60% acetonitrile and, about 5% water in combination thereof.

In certain embodiment, the mobile phase B comprises about 0.1% formic acid, and 99.9% water in combination thereof.

In an embodiment, the elution of non-ionic surfactant by using mobile phase A in substantially higher amount than mobile phase B, wherein the ratio of mobile phase A to mobile phase B maintained about 100:0, about 90:10, about 80:20, and about 70:30.

In an embodiment, the elution of protein of interest by using mobile phase B in substantially higher amount than mobile phase A, wherein the ratio of mobile phase B to mobile phase A maintained about 30:70, about 20:80, about 10:90, and about 0:100.

In an embodiment, the non-ionic surfactant is eluted when mobile phase A and mobile phase B ratio has substantial amount of mobile phase A.

In an embodiment, the ratio of mobile phase A to mobile phase B selected between 0:100 to about 100:0.

In an embodiment, the ratio of mobile phase B to mobile phase A selected between 0:100 to about 100:0.

In an embodiment, the ratio of mobile phase A to mobile phase B selected from about 100:0, about 10:90, about 90:10, about 0:100.

In an embodiment, the ratio of mobile phase B to mobile phase A selected from about 100:0, about 10:90, about 90:10, about 0:100.

In an embodiment, the temperature of ZIC-HILIC column is selected from about 10° C., about 15° C., about 20° C., about 25° C., about 30° C., about 35° C., about 40° C., about 45° C., about 50° C., about 55° C., about 60° C. and about 65° C.

In another embodiment, the temperature of ZIC-HILIC column is about 55° C. to about 65° C.

In an embodiment, the elution of non-ionic surfactant is performed at temperature about 55° C.

In certain embodiment, the separation and quantification of non-ionic surfactant in a protein mixture comprising protein of interest and non-ionic surfactant is performed at temperature about 55° C.

In an embodiment, the flow rate of mobile phase is selected from about 0.5 mL/min, about 0.10 mL/min, about 0.15 mL/min, about 0.20 mL/min, about 0.25 mL/min, about 0.30 mL/min, about 0.35 mL/min, about 0.40 mL/min, about 0.45 mL/min, about 0.50 mL/min, about 0.60 mL/min, about 0.65 mL/min, about 0.70 mL/min, about 0.75 mL/min, about 0.80 mL/min, about 0.85 mL/min, about 0.90 mL/min about 0.95 mL/min and about 1.0 mL/min.

In another embodiment, the flow rate of mobile phase is about 0.75±0.20 mL/min.

In an embodiment, the run time of biopharmaceutical formulation through ZIC-HILIC column is selected from about 0 to 50 minutes.

In another embodiment, the biopharmaceutical formulation run from about 0 to 45 minutes through ZIC-HILIC column.

In an embodiment, the non-ionic surfactant elutes from ZIC-HILIC column when mobile phase A is increased to 100%.

In an embodiment, the non-ionic surfactant elutes from ZIC-HILIC column when mobile phase A is increased to about 90%.

In an embodiment, the polypeptide elutes from ZIC-HILIC column when mobile phase A is reduced to about 90%.

In another embodiment, the polypeptide elutes from ZIC-HILIC column when mobile phase B is increased to about 90%.

In an embodiment, the pH of mobile phase is adjusted to pH selected from about pH 5 to about pH 8, about pH 6.3 to about pH 8, about pH 6.5 to about pH 8, and about pH 6.7 to about pH 8.

In another embodiment, the pH of mobile phase is adjusted to about pH 6.5.

In an embodiment, the non-ionic surfactant is separated and quantified using a ZIC-HILIC column, wherein the chromatogram peak gives the elution profile of non-ionic surfactant as early peak elutes before the elution of protein of interest.

In an embodiment, the method is very robust and provides a linearity, repeatability, and accuracy. The present disclosure provides a method that is required for the separation and quantification of non-ionic surfactant present in the biopharmaceutical formulation comprising antibody, proteins or fusion protein.

The present disclosure provides examples mentioned below for illustrative purpose and should not be consider limiting to them.

### Example 1—Identification of Non-Ionic Surfactant Polysorbate 20 (PS20) in Protein Sample Containing Anti-IgE Antibody

Details of Chromatographic parameters

| | |
|---|---|
| Column description | ZIC HILIC Column (250*4.6 mm, 5 μm, 200 Å) |
| Column temperature | 55° C. |
| Sample manager | 6° C. |
| temperature | |
| Flow rate | 0.75 mL/min |
| CAD Detector | Model Name: Corona Veo Charged Aerosol |
| | Detector |
| | Make: Thermo Fisher Scientific |
| | Cat No.: 5081.0010 |
| Mobile Phase | Mobile phase A: 0.1% FA in 35% Methanol, 60% |
| | Acetonitrile and 5% Water |
| | Mobile phase B: 0.1% FA in Water |

Chromatography gradient details

| Time | Mobile phase A | Mobile phase B | Flow |
|---|---|---|---|
| (min) | (%) | (%) | (mL/min) |
| 0 | 100 | 0 | 0.75 |
| 5 | 100 | 0 | |
| 15 | 10 | 90 | |
| 20 | 10 | 90 | |
| 23 | 100 | 0 | |
| 45 | 100 | 0 | |

### Steps:

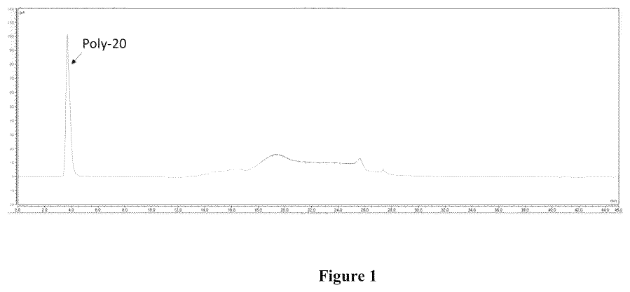

a) Polysorbate-20 stock solution (1 mg/mL) was prepared by weighing 10 mg of PS20 mixed using 60% acetonitrile in a 10 ml volumetric flask.
b) The concentration of PS20 working solution prepared from PS20 stock solution is 0.4 mg/mL in a 10 ml volumetric flask.
c) Diluent injected as blank run and there was not any peak observed within 8 minutes
d) PS20 injected and peak was observed around 3.6 minute in ZIC HILIC Column.
e) The biopharmaceutical formulation comprising antibody and polysorbate 20 standard injected into the ZIC HILIC Column and observation of prior void peak detected through CAD detector as shown in FIG. 1 and later other peaks were separated.
f) PS20 and biopharmaceutical formulation have different concentration, therefore peak height intensity is observed different in chromatogram as shown.
   Biopharmaceutical formulation contains 150 mg/ml IgE antibody, 0.4 mg/ml Polysorbate 20, L-arginine hydrochloride (42.1 mg/mL), L-histidine (1.37 mg/mL), L-histidine hydrochloride monohydrate (2.34 mg/mL).

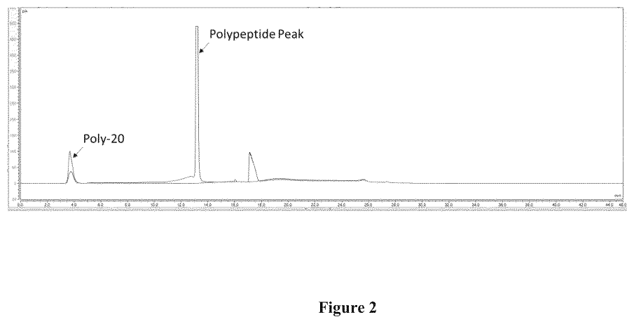

Results shown in FIG. 2 the separation and quantification profile as void peak of eluted non-ionic surfactant PS20 before the polypeptide peak by using a ZIC-HILIC column.

### Example 2—Specificity of Non-Ionic Surfactant Polysorbate 20 (PS20) in Protein Sample Containing Anti-IgE Antibody

Details of Chromatographic parameters

| | |
|---|---|
| Column description | ZIC HILIC Column (250*4.6 mm, 5 μm, 200 Å) |
| Column temperature | 55° C. |
| Sample manager | 15° C. |
| temperature | |
| Flow rate | 0.75 mL/min |
| CAD Detector | Model Name: Corona Veo Charged Aerosol |
| | Detector |
| | Make: Thermo Fisher Scientific |
| | Cat No.: 5081.0010 |
| Mobile Phase | Mobile phase A: 0.1% Formic Acid in 35% |
| | Methanol, 60% Acetonitrile and 5% Water |
| | Mobile phase B: 0.1% Formic Acid in Water |

Chromatography gradient details

| Time (min) | Flow (ml/min) | % A | % B |
|---|---|---|---|
| 0 | 0.75 | 100 | 0 |
| 5 | 0.75 | 100 | 0 |
| 15 | 0.75 | 10 | 90 |
| 20 | 0.75 | 10 | 90 |
| 23 | 0.75 | 100 | 0 |
| 45 | 0.75 | 100 | 0 |

### Steps:

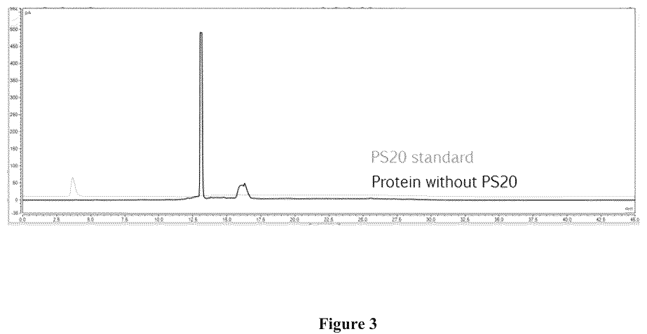

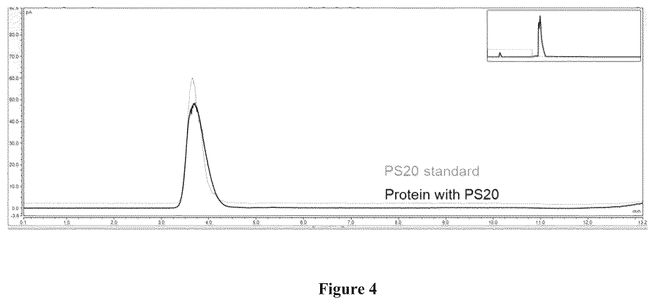

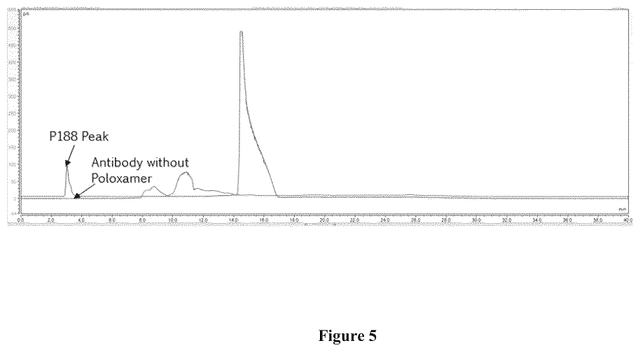

a) PS20 stock solution (2 mg/mL) was prepared by weighing about 100 mg PS20 and transferred into 50 mL of volumetric flask and water was added to make up final volume.
b) The concentration of PS20 working solution was prepared from PS20 stock solution is 0.14 mg/mL.
c) Protein sample containing anti-IgE antibody without PS20 was prepared by adding 50 μL sample and 50 μL water.
d) Protein sample containing anti-IgE antibody with PS20 was prepared by adding 60 μL sample and 60 μL water.
e) PS20 standard of 0.14 mg/mL and protein sample containing antibody without PS20 injected and peak was observed around 3.6 minute in standard while peak was not found in protein sample without PS20 as shown in FIG. 3.
f) PS20 standard of 0.14 mg/mL and protein sample containing antibody with PS20 injected and peak was observed around 3.6 minute in both standard and protein sample with PS20 as shown in FIG. 4.
   Protein sample contains 150 mg/ml IgE antibody, 0.4 mg/ml Polysorbate 20, L-arginine hydrochloride (42.1 mg/mL), L-histidine (1.37 mg/mL), L-histidine hydrochloride monohydrate (2.34 mg/mL).

### Example 3—Specificity of Non-Ionic Surfactant Poloxamer 188 (P188) in Protein Sample Containing Anti-IgE Antibody

Details of Chromatographic parameters

| | |
|---|---|
| Column description | ZIC HILIC Column (250*4.6 mm, 5 μm, 200 Å) |
| Column temperature | 55° C. |
| Sample manager | 15° C. |
| temperature | |
| Flow rate | 0.75 mL/min |
| CAD Detector | Model Name: Corona Veo Charged Aerosol |
| | Detector |
| | Make: Thermo Fisher Scientific |
| | Cat No.: 5081.0010 |
| Mobile Phase | Mobile phase A: 0.1% FA in 35% Methanol, 60% |
| | Acetonitrile and 5% Water |
| | Mobile phase B: 0.1% FA in Water |

Chromatography gradient details

| Time (min) | Flow (ml/min) | % A | % B |
|---|---|---|---|
| 0 | 0.75 | 90 | 10 |
| 5 | 0.75 | 90 | 10 |
| 15 | 0.75 | 10 | 90 |
| 20 | 0.75 | 10 | 90 |
| 23 | 0.75 | 90 | 10 |
| 40 | 0.75 | 90 | 10 |

### Steps:

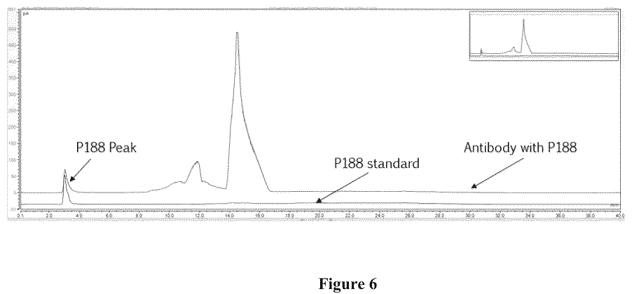

a) Poloxamer 188 stock solution (2 mg/mL) was prepared by weighing about 21.93 mg of Poloxamer 188, transferred into 10 mL of volumetric flask and water was added to make up final volume.
b) Poloxamer 188 working solution concentration 0.2 mg/mL was prepared using Poloxamer 188.
c) Protein sample anti-IgE antibody without Poloxamer 188 was prepared by mixing 50 μL sample and 50 μL water.
d) Protein sample containing anti-IgE antibody with Poloxamer 188 was prepared by mixing 60 μL sample and 60 μL water.
e) Poloxamer 188 standard of 0.2 mg/mL and protein sample containing antibody without Poloxamer 188 was injected and peak was observed around 3.1 minute in standard while peak was not found in protein sample containing anti-IgE antibody without Poloxamer 188 as shown in FIG. 5.
f) Poloxamer 188 standard of 0.2 mg/mL and protein sample containing antibody with Poloxamer 188 was injected and peak was observed around 3.1 minute in both standard and protein sample containing antibody with Poloxamer 188 as shown in FIG. 6.
   Protein sample contains 150 mg/ml anti-IgE antibody, 0.4 mg/ml Poloxamer 188, L-arginine hydrochloride (200 mM), Sodium Phosphate monobasic and dibasic (20 mM).

### Example 4—Specificity of Non-Ionic Surfactant Poloxamer 188 (P188) in Protein Sample Containing CTLA4-Ig Fusion Protein

Details of Chromatographic parameters

| | |
|---|---|
| Column description | ZIC HILIC Column (250*4.6 mm, 5 μm, 200 Å) |
| Column temperature | 55° C. |
| Sample manager | 15° C. |
| temperature | |
| Flow rate | 0.75 mL/min |
| CAD Detector | Model Name: Corona Veo Charged Aerosol |
| | Detector |
| | Make: Thermo Fisher Scientific |
| | Cat No.: 5081.0010 |
| Mobile Phase | Mobile phase A: 0.1% Formic Acid in 35% |
| | Methanol, 60% Acetonitrile and 5% Water |
| | Mobile phase B: 0.1% Formic Acid in Water |

Chromatography gradient details

| Time (min) | Flow (ml/min) | % A | % B |
|---|---|---|---|
| 0 | 0.75 | 90 | 10 |
| 5 | 0.75 | 90 | 10 |
| 15 | 0.75 | 10 | 90 |
| 20 | 0.75 | 10 | 90 |
| 23 | 0.75 | 90 | 10 |
| 40 | 0.75 | 90 | 10 |

### Steps:

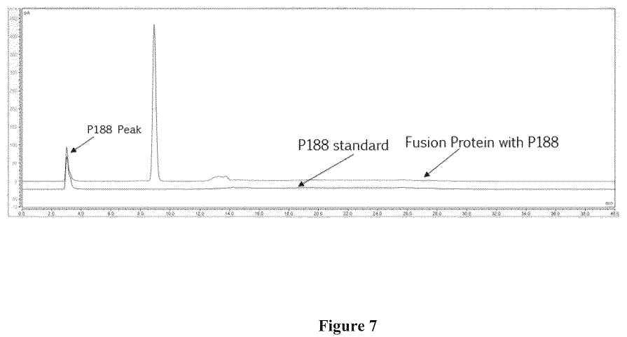

a) Poloxamer 188 stock solution (2 mg/mL) was prepared by weighing about 21.93 mg Poloxamer 188, transferred into 10 mL of volumetric flask the water was added to make up final volume.
b) Poloxamer 188 working solution (0.2 mg/mL) was prepared from Poloxamer 188 stock solution.
c) The protein sample containing fusion protein sample with Poloxamer 188 was prepared by mixing 5 μL sample and 195 μL 60% acetonitrile (ACN). The concentration of Poloxamer 188 (8 mg/mL), was diluted to obtain 0.2 mg/mL poloxamer in sample.
d) Poloxamer 188 standard of 0.2 mg/mL and protein sample containing fusion protein with Poloxamer 188 injected and peak was observed around 3.1 minute in both standard and protein sample containing fusion protein with Poloxamer 188 as shown in FIG. 7.
   Protein sample contains 125 mg/ml CTLA4-Ig fusion protein, poloxamer 188 (8 mg/mL), dibasic sodium phosphate anhydrous (0.838 mg/mL), monobasic sodium phosphate monohydrate (0.286 mg/mL), and sucrose (170 mg/mL).

### Example 5—Effect of Different Column Temperature on Non-Ionic Surfactant Poloxamer 188 (P188) Peak

Details of Chromatographic parameters

| | |
|---|---|
| Column description | ZIC HILIC Column (250*4.6 mm, 5 μm, 200 Å) |
| Column temperature | 45° C., 55° C. and 60° C. |
| Sample manager | 15° C. |
| temperature | |
| Flow rate | 0.75 mL/min |
| CAD Detector | Model Name: Corona Veo Charged Aerosol |
| | Detector |
| | Make: Thermo Fisher Scientific |
| | Cat No.: 5081.0010 |
| Mobile Phase | Mobile phase A: 0.1% Formic Acid in 35% |
| | Methanol, 60% Acetonitrile and 5% Water |
| | Mobile phase B: 0.1% Formic Acid in Water |

Chromatography gradient details

| Time (min) | Flow (ml/min) | % A | % B |
|---|---|---|---|
| 0 | 0.75 | 90 | 10 |
| 5 | 0.75 | 90 | 10 |
| 15 | 0.75 | 10 | 90 |
| 20 | 0.75 | 10 | 90 |
| 23 | 0.75 | 90 | 10 |
| 40 | 0.75 | 90 | 10 |

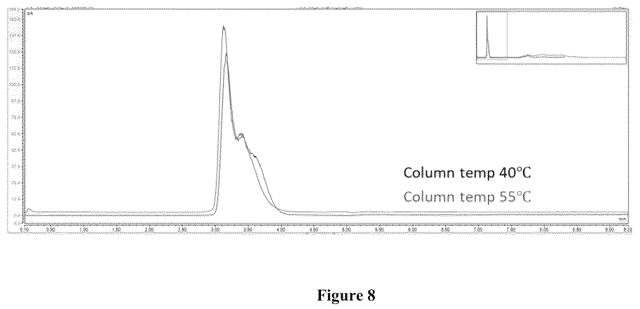

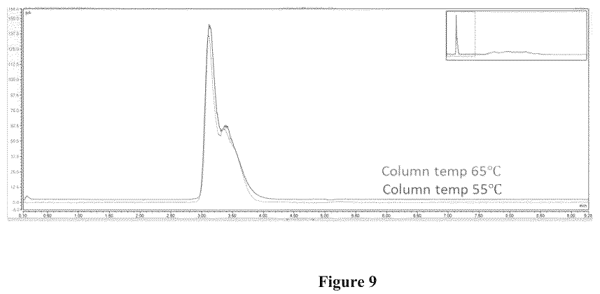

a) Poloxamer 188 stock solution (2 mg/mL) was prepared by weigh about 20 mg Poloxamer, transferred into 10 mL of volumetric flask and water was added to make up final volume.
b) Poloxamer 188 (0.4 mg/mL) solution was prepared by addition of 200 μL of 2 mg/ml Poloxamer 188 stock solution and 800 μl milli Q water.
c) The overlay of Poloxamer 188 peak using different column temperature 40° C. and 55° C. as shown in FIG. 8 as Peak width increased when temperature decreased, while column temperature 55° C. and 65° C. was found suitable as shown in FIG. 9.

### Example 6—Linearity, Repeatability, and Accuracy of Non-Ionic Surfactant Poloxamer 188

Details of Chromatographic parameters

| | |
|---|---|
| Column description | ZIC HILIC Column (250*4.6 mm, 5 μm, 200 Å) |
| Column temperature | 55° C. |
| Sample manager | 15° C. |
| temperature | |
| Flow rate | 0.75 mL/min |
| CAD Detector | Model Name: Corona Veo Charged Aerosol |
| | Detector |
| | Make: Thermo Fisher Scientific |
| | Cat No.: 5081.0010 |
| Mobile Phase | Mobile phase A: 0.1% FA in 35% Methanol, 60% |
| | Acetonitrile and 5% Water |
| | Mobile phase B: 0.1% FA in Water |

Chromatography gradient details

| Time (min) | Flow (ml/min) | % A | % B |
|---|---|---|---|
| 0 | 0.75 | 90 | 10 |
| 5 | 0.75 | 90 | 10 |
| 15 | 0.75 | 10 | 90 |
| 20 | 0.75 | 10 | 90 |
| 23 | 0.75 | 90 | 10 |
| 40 | 0.75 | 90 | 10 |

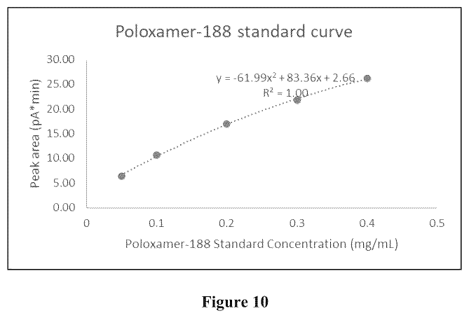

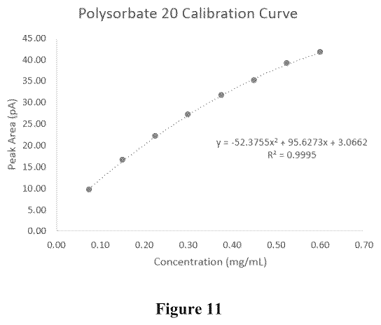

The Linearity was determined by following the steps as below:
a) Linearity was prepared from 0.05 mg/mL to 0.40 mg/mL by using stock solution of 2 mg/mL.
b) Each linearity solution was prepared in triplicate, injected and results as shown in Table 1 and Linearity curve (Quadratic calibration curve) between average peak area (pA*min) and concentration (mg/mL) as shown in FIG. 10.
c) Method was observed linear from 0.05 to 0.40 mg/mL Poloxamer concentration. The coefficient of determination (R²) of the regression was 1.00. Precision in linearity range (0.05 to 0.40 mg/mL) was found between 1.0% to 5.7% and accuracy was found between 92% to 108%.

TABLE 1
Results for Linearity

| | | | Average |
|---|---|---|---|
| | Concentration | Area | Area |
| Standard solution Name | (mg/mL) | (pA*min) | (pA*min) |
| 0.05 mg/mL Poloxamer standard | 0.05 | 6.3740 | 6.4097 |
| preparation 1 | | | |
| 0.05 mg/mL Poloxamer standard | 0.05 | 6.3910 | |
| preparation 2 | | | |
| 0.05 mg/mL Poloxamer standard | 0.05 | 6.4640 | |
| preparation 3 | | | |
| 0.10 mg/mL Poloxamer standard | 0.10 | 10.7780 | 10.7223 |
| preparation 1 | | | |
| 0.10 mg/mL Poloxamer standard | 0.10 | 10.6570 | |
| preparation 2 | | | |
| 0.10 mg/mL Poloxamer standard | 0.10 | 10.7320 | |
| preparation 3 | | | |
| 0.20 mg/mL Poloxamer standard | 0.20 | 16.8430 | 16.9447 |
| preparation 1 | | | |
| 0.20 mg/mL Poloxamer standard | 0.20 | 16.7620 | |
| preparation 2 | | | |
| 0.20 mg/mL Poloxamer standard | 0.20 | 17.2290 | |
| preparation 3 | | | |
| 0.30 mg/mL Poloxamer standard | 0.30 | 21.5900 | 21.7653 |
| preparation 1 | | | |
| 0.30 mg/mL Poloxamer standard | 0.30 | 21.8490 | |
| preparation 2 | | | |
| 0.30 mg/mL Poloxamer standard | 0.30 | 21.8570 | |
| preparation 3 | | | |
| 0.40 mg/mL Poloxamer standard | 0.40 | 25.7060 | 26.2250 |
| preparation 1 | | | |
| 0.40 mg/mL Poloxamer standard | 0.40 | 25.8830 | |
| preparation 2 | | | |
| 0.40 mg/mL Poloxamer standard | 0.40 | 27.0860 | |
| preparation 3 | | | |

The repeatability was determined by following the steps as below:
a) Poloxamer 188 was spiked in protein samples (anti-IgE antibody). Individually six samples were prepared with protein sample (anti-IgE antibody) that does not contain Poloxamer 188 or any other surfactant priorly.
b) 80 μL Poloxamer-188 was spiked in 320 μL protein sample (anti-IgE antibody) without Poloxamer 188 and mixed.
c) 400 μL Acetonitrile was added and vortex at maximum speed for about two minutes.
d) Spinned at maximum speed for 10 minutes at temperature around 5° C. Supernatant was injected.
e) Repeatability of Poloxamer 188 concentration (n=6) was found 3.7% as shown in Table 2.

TABLE 2
Results for repeatability

| | | Concentration | |
|---|---|---|---|
| | Area | back calculated | |
| Sample Name | (pA*min) | (mg/mL) | % RSD |
| Preparation-1 | 15.598 | 0.199 | 3.7% |
| Preparation-2 | 15.842 | 0.203 | |
| Preparation-3 | 16.765 | 0.221 | |
| Preparation-4 | 15.868 | 0.204 | |
| Preparation-5 | 16.029 | 0.207 | |
| Preparation-6 | 16.096 | 0.208 | |

The accuracy was determined by following the steps as below:
a) Poloxamer 188 was spiked in protein samples (anti-IgE antibody) at 0.5 μg (0.1 mg/mL), 1.0 μg (0.2 mg/mL) and 1.5 μg (0.3 mg/mL).
b) Acetonitrile was added to precipitate out protein and then supernatant injected. Protein samples (anti-IgE antibody) doesn't contain Poloxamer 188 or any other surfactant priorly. Three individual samples were prepared.
c) Based on the observation the accuracy was observed at concentrations of 0.10 mg/mL, 0.20 mg/mL and 0.30 mg/mL where % recovery was found within 80-120% and maximum % RSD (Relative standard deviation) was observed 5.5 for 0.2 mg/mL spiking in Protein samples (anti-IgE antibody) as shown in Table 3.

TABLE 3
Results for accuracy of sample

| | | | Amount | % | |
|---|---|---|---|---|---|
| | Amount | | back | Re- | |
| | Loaded | Area | calculated | cov- | % |
| Sample Name | (mg/mL) | (pA*min) | (mg/mL) | ery | RSD |
| Protein + 0.10 mg/mL | 0.10 | 9.456 | 0.093 | 93 | 2.2 |
| spiked Poloxamer | | | | | |
| Preparation 1 | | | | | |
| Protein + 0.10 mg/mL | 0.10 | 9.531 | 0.094 | 94 | |
| spiked Poloxamer | | | | | |
| Preparation 2 | | | | | |
| Protein + 0.10 mg/mL | 0.10 | 9.669 | 0.097 | 97 | |
| spiked Poloxamer | | | | | |
| Preparation 3 | | | | | |
| Protein + 0.20 mg/mL | 0.20 | 15.436 | 0.189 | 95 | 5.5 |
| spiked Poloxamer | | | | | |
| Preparation 1 | | | | | |
| Protein + 0.20 mg/mL | 0.20 | 16.022 | 0.199 | 100 | |
| spiked Poloxamer | | | | | |
| Preparation 2 | | | | | |
| Protein + 0.20 mg/mL | 0.20 | 16.642 | 0.211 | 106 | |
| spiked Poloxamer | | | | | |
| Preparation 3 | | | | | |
| Protein + 0.3 mg/mL | 0.30 | 19.881 | 0.273 | 91 | 3.9 |
| spiked Poloxamer | | | | | |
| Preparation1 | | | | | |
| Protein + 0.3 mg/mL | 0.30 | 20.402 | 0.284 | 95 | |
| spiked Poloxamer | | | | | |
| Preparation 2 | | | | | |
| Protein + 0.3 mg/mL | 0.30 | 20.874 | 0.295 | 98 | |
| spiked Poloxamer | | | | | |
| Preparation 3 | | | | | |

### Example 7—Linearity, Repeatability, and Accuracy of Non-Ionic Surfactant Polysorbate 20

Details of Chromatographic parameters

| | |
|---|---|
| Column description | ZIC HILIC Column (250*4.6 mm, 5 μm, 200 Å) |
| Column temperature | 55° C. |
| Sample manager | 5° C. |
| temperature | |
| Flow rate | 0.75 mL/min |
| CAD Detector | Model Name: Corona Veo Charged Aerosol |
| | Detector |
| | Make: Thermo Fisher Scientific |
| | Cat No.: 5081.0010 |
| Mobile Phase | Mobile phase A: 0.1% Formic acid in 35% |
| | Methanol, 60% Acetonitrile and 5% Water |
| | Mobile phase B: 0.1% Formic acid in Water |

Chromatography gradient details

| Time (min) | Flow (ml/min) | % A | % B |
|---|---|---|---|
| 0 | 0.75 | 100 | 0 |
| 5 | 0.75 | 100 | 0 |
| 15 | 0.75 | 10 | 90 |
| 20 | 0.75 | 10 | 90 |
| 23 | 0.75 | 100 | 0 |
| 45 | 0.75 | 100 | 0 |

The linearity and accuracy were determined by following the steps as below:
a) Linearity was prepared from 0.075 mg/mL to 0.60 mg/mL using stock solution of 2 mg/mL Polysorbate 20.
b) Each linearity solution was prepared in triplicate and injected.
c) Method was observed linear from 0.075 to 0.60 mg/mL Polysorbate 20 concentration as in Table 4 and FIG. 11.
d) The coefficient of determination (R2) of the regression was 1.00 and accuracy was found between 96% to 102%.

TABLE 4
Results for linearity of sample

| | | | | | | Back | |
|---|---|---|---|---|---|---|---|
| | | Preparation- | Preparation- | Preparation- | Average | calculated | |
| Linearity | Concentration | 1 Area | 2 Area | 3 Area | Area | Concentration | |
| solution | (mg/mL) | (pA) | (pA) | (pA) | (pA) | (mg/mL) | % Recovery |
| L1 | 0.075 | 9.9702 | 9.5978 | 9.4859 | 9.6846 | 0.0721 | 96 |
| L2 | 0.150 | 16.6651 | 16.3440 | 16.4043 | 16.4711 | 0.1530 | 102 |
| L3 | 0.225 | 22.3743 | 22.0705 | 21.9691 | 22.1380 | 0.2279 | 101 |
| L4 | 0.300 | 27.3779 | 27.0384 | 26.8891 | 27.1018 | 0.3010 | 100 |
| L5 | 0.375 | 31.8127 | 31.3966 | 31.3752 | 31.5282 | 0.3744 | 100 |
| L6 | 0.450 | 35.2900 | 35.0321 | 34.6768 | 34.9996 | 0.4399 | 98 |
| L7 | 0.525 | 39.4340 | 39.0361 | 38.5730 | 39.0144 | 0.5295 | 101 |
| L8 | 0.600 | 43.0256 | 41.9627 | 40.0645 | 41.6843 | 0.6030 | 100 |

The repeatability was determined by following the steps as below:
a) Six individual solution 0.14 mg/mL standard of PS20 were prepared from the stock solution of 2 mg/mL and injected.
b) Results for repeatability as shown in Table 5.
c) 0.56% RSD of area (n=6) was observed of six individual preparations of standards.

TABLE 5
Results for repeatability of sample

| Sample Name | Area (pA*min) | % RSD of area |
|---|---|---|
| Preparation-1 | 25.8857 | 0.56 |
| Preparation-2 | 25.7186 | |
| Preparation-3 | 25.7481 | |
| Preparation-4 | 25.4731 | |
| Preparation-5 | 25.6336 | |
| Preparation-6 | 25.5772 | |

Dated this 31 Dec. 2022.
Signature: Digitally signed by Kaushal Joshi
Director—IP

1. A method for separation of non-ionic surfactant from a protein mixture comprising protein of interest and non-ionic surfactant comprising;
a) loading the protein mixture on zwitterionic hydrophilic interaction liquid chromatography (ZIC-HILIC) column;
b) eluting the non-ionic surfactant from ZIC-HILIC column by using mobile phase A and B in a suitable ratio;
c) eluting the protein of interest from ZIC-HILIC column by using mobile phase A and B in a suitable ratio;
wherein the protein mixture is a biopharmaceutical formulation or composition, and the non-ionic surfactant elutes before the elution of protein of interest.

2. The method as claimed in claim 1, wherein the eluted non-ionic surfactant in step (b) quantified by suitable technique selected from ultraviolet light absorbance (UV), fluorescence (FL), refractive index (RI), evaporative light scattering (ELSD), charged aerosol (CAD), and mass spectrometry (MS).

3. The method as claimed in claim 1, wherein the non-ionic surfactant selected from golyglycerol alkyl ethers, glucosyl dialkyl ethers, crownethers, ester-linked surfactants, polyoxyethylene alkyl ethers, BYK-110, Polysorbate 20 (PS 20), Polysorbate 40 (PS 40), Polysorbate (PS 60), Polysorbate 80 (PS 80), Poloxamer 188 (P188), Poloxamer 237 (P237), Poloxamer (P338), Poloxamer (P407), and Nonidet-P40 (nonylphenoxypolyethoxyethanol).

4. The method as claimed in claim 3, wherein non-ionic surfactant is Polysorbate 20 (PS20), Poloxamer 188 (P188).

5. The method as claimed in claim 1, wherein mobile phase A or mobile phase B is an organic solvent, or water and/or combination thereof to improve the binding affinity of protein of interest with column resin.

6. The method as claimed in claim 5, wherein methanol, ethanol, acetic acid, trifluoroacetic acid, isopropanol.

7. The method as claimed in claim 6, wherein mobile phase A or mobile phase B is an organic solvent, or water and/or combination thereof to improve the elution of protein of interest.

8. The method as claimed in claim 5, wherein mobile phase A is an organic solvent, or water and/or combination thereof and mobile phase B is formic acid and water and/or combination thereof.

9. The method as claimed in claim 1, wherein the protein of interest is selected from peptide, antibody, antibody fragment, PEGylated protein, and fusion protein.

10. The method as claimed in claim 9, wherein the antibody or fusion protein is selected from Rituximab, Palivizumab, Etanercept, Abatacept, Aflibercept, Belatacept, Rilonacept, Romiplostim, Alefacept, Conbercept, Infliximab, Trastuzumab, Alemtuzumab, Adalimumab, Ibritumomab tiuxetan, Omalizumab, Cetuximab, Bevacizumab, Natalizumab, Eculizumab, Certolizumab pegol, Ustekinumab, Canakinumab, Golimumab, Ofatumumab, Tocilizumab, Denosumab, Belimumab, Ipilimumab, Brentuximab vedotin, Pertuzumab, Trastuzumab emtansine, Raxibacumab, Obinutuzumab, Siltuximab, Ramucimmab, Vedolizumab, Blinatumomab, Nivolumab, Pembrolizumab, Darucizumab, Necitumumab, Dinutuximab, Secukinumab, Mepolizumab, Alirocumab, Evolocumab, Daratumumab, Elotuzumab, Ixekizumab, Reslizumab, Olaratumab, Bezlotoxumab, Atezolizumab, Obiltoxaximab, Sarilumab, Ocrelizumab, Tildrakizumab, Romosozumab, Brolucizumab, and Crizanlizumab.

11. The method as claimed in claim 6, wherein the concentration of formic acid is selected from about 0.1%, about 0.2%, about 0.3%, about 0.4%, about 0.5%, about 0.6%, about 0.7%, about 0.8%, about 0.9% and about 1%.

12. The method as claimed in claim 11, wherein the concentration of formic acid is about 0.1%.

13. The method as claimed in claim 6, wherein the concentration of methanol is selected from about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%.

14. The method as claimed in claim 13, wherein the concentration of methanol is about 35%.

15. The method as claimed in claim 6, wherein the concentration of acetonitrile is selected from about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%.

16. The method as claimed in claim 15, wherein the concentration of acetonitrile is about 60%.

17. The method as claimed in claim 5, wherein the concentration of water is selected from about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97% about 98%, and 99.9%.

18. The method as claimed in claim 17, wherein the concentration of water is about 5% or 99.9%.

19. The method as claimed in claim 1, wherein the elution of non-ionic surfactant in step (b) is performed by using mobile phase A in substantially higher amount than mobile phase B, wherein the ratio of mobile phase A to mobile phase B is selected from about 100:0, about 90:10, about 80:20, and about 70:30.

20. The method as claimed in 1, wherein the elution of protein of interest in step (c) by using mobile phase B in substantially higher amount than mobile phase A, wherein the ratio of mobile phase B to mobile phase A is selected from about 30:70, about 20:80, about 10:90, and about 0:100.

21. A method for improving the resolution of non-ionic surfactant in protein mixture comprising;
a) loading the protein mixture comprising protein of interest and non-ionic surfactant on zwitterionic hydrophilic interaction liquid chromatography (ZIC-HILIC) column;
b) eluting the non-ionic surfactant from the zwitterionic hydrophilic interaction liquid chromatography (ZIC-HILIC) column;
wherein the elution of non-ionic surfactant is performed at temperature more than about 50° C. to about 65° C.

22. The method as claimed in 1, wherein the elution of non-ionic surfactant is performed at temperature about 55° C.

23. The method as claimed in claim 7, wherein mobile phase A is an organic solvent, or water and/or combination thereof and mobile phase B is formic acid and water and/or combination thereof.

24. The method as claimed in claim 8, wherein the concentration of formic acid is selected from about 0.1%, about 0.2%, about 0.3%, about 0.4%, about 0.5%, about 0.6%, about 0.7%, about 0.8%, about 0.9% and about 1%.

25. The method as claimed in claim 24, wherein the concentration of formic acid is about 0.1%.

26. The method as claimed in claim 7, wherein the concentration of water is selected from about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97% about 98%, and 99.9%.

27. The method as claimed in claim 23, wherein the concentration of water is about 5% or 99.9%.

28. The method as claimed in claim 8, wherein the concentration of water is selected from about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97% about 98%, and 99.9%.

29. The method as claimed in claim 28, wherein the concentration of water is about 5% or 99.9%.
